(19) United States
(12) Patent Application Publication
(10) Pub. No.: US 2017/0183644 A1
(43) Pub. Date: Jun. 29, 2017

(54) DRUG COCKTAIL ANALYSES USING MICROSCALE VORTEX ASSISTED ELECTROPORATION

(71) Applicant: President and Fellows of Harvard College, Cambridge, MA (US)

(72) Inventors: Dwayne A. Vickers, Cambridge, MA (US); Soojung Claire Hur, Malden, MA (US)

(21) Appl. No.: 15/406,210

(22) PCT Filed: Jul. 14, 2015
(86) PCT No.: PCT/US15/40422

§ 371 Date: Jan. 13, 2017

Related U.S. Application Data
(60) Provisional application No. 62/047,317, filed on Sep. 8, 2014.

(51) Int. Cl. C12N 13/00; C12Q 1/18; G01N 33/50

(52) U.S. Cl. CPC C12N 13/00; G01N 33/5011; C12Q 1/18

(57) ABSTRACT

A vortex flow based method for electroporating molecules, e g., sequentially, into cells is provided. The invention provides a method to determine the effect of two or more molecules of interest on cells. The method comprises maintaining a vortex flow in multiple traps having cells of interest; providing a first molecule of interest to the traps and an electric field across the traps for a defined first period of time; providing a second molecule of interest to the traps and an electric field across the traps for a defined second period of time; and determining the combined effect of the first and second molecules on the cells, e.g., whether the combined effect of the first and second molecules on the cells is synergistic, neutral or antagonistic.

### CROSS-REFERENCE TO RELATED APPLICATIONS

This application claims the benefit of the filing date of U.S. application Ser. No. 62/024,317, filed on Jul. 14, 2014, the disclosure of which is incorporated by reference herein.

### BACKGROUND

Combination drug therapy became a promising approach for treating complex diseases such as cancer (Meng et al., 2010), HIV (Mendez-Ortega et al., 2010), cardiovascular disease (Williams, 2003) and type-2 diabetes (Wu et al., 2010). For such therapy, two or more already-licensed drugs are simultaneously administered to maximize therapeutic drug efficacy by targeting multiple signaling pathways while minimizing overlapping toxicity and inhibiting resistance developing mechanisms (Xu et al., 2012; Chou, 2016; Kolishetti et al., 2010). Due to its therapeutic benefits, extensive efforts have focused on the discovery of new drug combinations that work synergistically. In order for newly discovered drug combinations to rapidly progress to the clinic, systematic unbiased drug screening strategies should complement existing hypothesis-driven approaches (Al-Lozikan et al., 2012). However, identification of respective doses for individual agents that induce synergistic therapeutic outcomes is an essential but challenging task. Consequently, these drug combination screenings are very laborious, thus the use of high throughput liquid dispensing systems are inevitable for processing time reduction and precision improvement (Booth et al, 2009). However, not only have such systems high fixed and operating costs (Du et al., 2013) but their assay performances also are susceptible to noises and variations inherent in cell-based microplate assays (Lundholt et al., 2003). Furthermore, a real dose-response curve and the cytotoxicity of drug combinations are difficult to measure if drugs have widely varying physicochemical properties, yielding inconsistent transport across the cell membrane.

Transient and reversible cellular membrane permeabilization utilizing electric pulses, namely electroporation, can be an appealing means for drug screening applications. It permits direct injections of newly developed cytotoxic molecules, which are inherently membrane-impermeable but can be subsequently modified to facilitate intracellular transport (Cemazar et al., 2001; Nakamura et al., 2013; Orlowski et al, 1988). In particular, electroporation has been used to enhance chemotherapeutic drug efficacy for cutaneous cancers (Gothelf et al, 2003; Todoronc et al., 2009; Jaroszeski et al., 2000).

### SUMMARY OF THE INVENTION

Combination therapy has become one of the leading approaches for treating complex diseases because it co-administers clinically proven drugs to target multiple signaling pathways of diseased cells. Identification of synergic drug combinations at their respective effective doses without unwanted accumulative side effects is the key to success for such therapy. As described herein, vortex-assisted microfluidic electroporation system was employed for direct drug cocktail analyses where drug substances were individually delivered into cytosols in a sequential and dosage-controlled manner. Through quantitative analyses, the synergic combinational dosage ratios of the chemotherapeutic drug and the anti-cancer flavonoid were identified. When optionally integrated with high-throughput label-free rare cell purification techniques, the present system allows for development of personalized medicines as the system would be capable of comprehensively assessing drug combinations directly on patients' cellular samples.

The invention provides a method to determine the effect of two or more molecules of interest on cells, e.g., to determine a combination index (CI). The method comprises maintaining a vortex flow in multiple traps having cells of interest; providing a first molecule of interest to the traps and an electric field across the traps for a defined first period of time; providing a second molecule of interest to the traps and an electric field across the traps for a defined second period of time; and determining the combined effect of the first and second molecules on the cells, e.g., whether the combined effect of the first and second molecules on the cells is synergistic, neutral or antagonistic. The periods of time the electric field is applied correlate to amounts or concentrations of the molecule(s) delivered to the cells. The combined effect of the molecules may also be determined at one or more different amounts or concentrations of the first molecule, the second molecule, or both molecules. The combined effect may be compared to the effect of the first molecule on the cells (e.g., for the first period of time but without the second molecule), to the effect of the second molecule (for the second period of time without the first molecule), or both. In one embodiment, a combination index (CI) is determined. For example, a CI<1 is synergistic and a CI>1 is antagonistic. In one embodiment, the CI is 0.85 to 0.9, 0.7 to 0.85; 0.3 to 0.7, 0.1 to 0.3 or <0.1. In one embodiment, periods of time and/or amounts or concentrations of the first and second molecules are identified that provide for synergistic effects, i.e., an effect that is more than additive. Synergistic interactions amongst drug combinations are highly desirable and sought after since they can result in increased efficacy, decreased dosage, reduced side toxicity, and/or minimized development of resistance for the patient. Thus, a dose delivered to a patient may be adjusted to result in increased efficacy, reduced side toxicity, and/or minimized development of resistance based on the methods of the invention. Combinations or amounts of drugs that are antagonistic may also be avoided using the methods of the invention.

In one embodiment, cells of interest are transported in a first fluid solution via a channel at a speed such that the fluid has a Reynolds number of greater than 100 to create the vortex flow in the traps. In one embodiment, a second fluid solution containing the first molecule of interest is provided to the traps while maintaining the vortex flow in the traps and after removing the first solution. In one embodiment, the electric field across the traps is substantially uniform. In one embodiment, electric flow across different traps is not uniform, e.g., cells in traps along different channels may be subjected to different electric fields. In one embodiment, a third fluid solution containing a second molecule of interest is provided to the traps while maintaining the vortex flow in the traps and after removing the solution having the first molecule of interest. Other solutions may be introduced, e.g., for washing, while maintaining the vortex flow. In one embodiment, the vortex flow is maintained until cells are collected for further analysis. In one embodiment, an inertial focusing region causes the cells of interest to move close to the sides of the traps via fluidic forces. In one embodiment, multiple channels are employed, each having opposing pairs of traps disposed along a length of the channels, and optionally having distinct inertial focusing regions and/or electrodes that deliver the electric field, e.g., ones that can be controlled independently. In one embodiment, the sample to be analyzed is a physiological fluid sample having cells of interest. In one embodiment, the cells of interest are cancer cells, e.g., melanoma cells or non-small cell lung cancer cells. In one embodiment, the cells are primary cells. In one embodiment, the cells are from a patient. In one embodiment, the cells are stem cells. In one embodiment, the cells are induced pluripotent stem cells. In one embodiment, the cells are circulating tumor cells that are isolated from a physiological fluid, e.g., blood, urine, peritoneal fluid or pleural fluid (see, e.g., U.S. 2013/0171628, the disclosure of which is incorporated by reference herein). In one embodiment, the first or second molecule is a chemotherapeutic drug, a drug to inhibit or treat cardiovascular disease, a drug to inhibit or treat diabetes, an anti-viral drug, an anti-bacterial drug, or an anti-parasitic drug. In one embodiment, the first or the second molecule is a MEK inhibitor, e.g., GSK 1120212, a BRAF inhibitor, e.g., PLX 4032, an ERK inhibitor, e.g., SCH772984, or EGFR kinase inhibitor, e.g., gefitinib and erolotinib. In one embodiment, at least one of the molecules of interest is not nucleic acid. Further provided is a system. The system includes a plurality of serial opposed pairs of traps disposed along a length of a first channel downstream of a first inertial focusing region, the size of each trap adapted to promote vortex flow within the traps while a solution is flowing through the first channel; a plurality of serial opposed pairs of traps disposed along a length of a second channel downstream of a second inertial focusing region, the size of each trap adapted to promote vortex flow within the traps while a solution is flowing through the second channel; wherein the first inertial focusing region and the second inertial focusing region each move cells in a solution travelling through the first channel and the second channel towards sides of the traps; an outlet for the first channel and the second channel disposed downstream from the plurality of serial opposed pairs of traps; and electrodes coupled to ends of the opposed traps to apply an electric field across the traps suitable for electroporation of molecules into the cells. In one embodiment, the channel has a width such that cells traveling through the channel are approximately 30 percent or greater than the width of the channel. In one embodiment, the inertial focusing region of the channel is approximately 0.7 cm or longer. In one embodiment, a second channel configured the same as the first channel is in parallel with the first channel. In one embodiment, the system includes 3 or more channels, e.g., 4, 5, 10, 15, 16, 20 or more channels. In one embodiment, the system further comprises multiple inlets to the first and second channel inertial focusing regions, the inlets adapted to receive solutions from multiple sources including a cell solution and a molecule solution. In one embodiment, the inlets are adapted to receive further solutions for incubation and flushing. In one embodiment, the channels each individually include opposing pairs of traps, e.g., 1, 2, 5, 10, 12, 15 or more opposing pairs of traps, serially disposed downstream from the inertial focusing region of each channel. In one embodiment, each trap is approximately square in shape with each opposed pair of traps forming a rectangle having a channel entrance and exit bisecting the opposed pair of traps. In one embodiment, each trap is approximately oval in shape with each opposed pair of traps having a channel entrance and exit bisecting the opposed pair of traps. However, traps that are not approximately rectangular or oval in shape are also envisioned. In one embodiment, the traps have sides (lengths) of between approximately 1 mm to 500 μm, 1 mm to 400 μm, 2 mm to 400 μm, or 1 mm to 300 μm. In one embodiment, the electrodes comprise multiple electrode tips adapted to create a substantially uniform DC or AC electric field, and wherein traps from the first channel adjacent traps from the second channel share an electrode of one polarity. For example: If DC is used, the pulse condition is 100V, 30 ms square pulse, every 2 second apart. An AC example may include a 100 Vrms, 10 kHz signal for 30 ms, every 2 seconds. It is envisioned that electrodes may be in parallel or in series so long as they are aligned for proper polarity. In one embodiment, the methods may be practiced in a system as described above.

### BRIEF DESCRIPTION OF THE FIGURES

FIG. 1. (A) Photograph of the microfluidic electroporation system used for sequential molecular delivery to perform direct drug cocktail analysis. The device consists of inlets for cells, drugs and a flush solution, two straight inertial focusing channels, ten electroporation chambers with electrodes and an outlet. The length (L_{c}) and the width (W_{c}) of individual electroporation chambers are 840 μm and 400 μm, respectively. (B) The representative fluorescent microscopic image illustrating parallel, dual-molecular delivery into trapped cell populations. Green and red fluorescent signals indicate successful penetration of two nucleic acid dyes, PI and YOYO®-1, respectively. Image contrast has been enhanced by adjusting the look-up table (LUT).

FIG. 2. Dose response curves of MDA-MB-231 cells to (A) gemcitabine (GMB), (B) bleomycin (BLM), (C) topotecan (TOP) and (D) quercetin alone. For all tested chemotherapeutic drugs, GMB, BLM, and TOP, viability of MDA-MB-231 cells substantially decreased with increasing exposure drug concentrations while the effect of quercetin as a single agent on cell viability was negligible and dose independent. Cells processed with the microfluidic assay exhibited stronger dose response than those treated using the conventional assay for all tested chemotherapeutic drugs. The proposed microfluidic assay can be used to identify effectiveness of drugs with subtle variation in the concentrations below IC₅₀ of 2 mM, 30 and 2 μM for GEM, BLM and TOP, respectively. The viability of electroporated cells without drug-treatment is presented for comparison (blue square). Asterisk indicates p<0.001. Error bar represents the standard error of measurements from three independent experiments.

FIG. 3: Dose-effect for topotecan-quercetin combination for MDA-MB-231 cells. (A) The anti-cancer effects of topotecan (30 second exposure post-electroporation, equivalent to 330 nM) in highly invasive metastatic breast cancer cells were enhanced with increasing quercetin dosages for both assays. (B) When the concentration of quercetin for the assay was fixed at 500 nM (equivalent to 30 second exposure post-electroporation), cells processed with the microfluidic assay exhibited sensitive dose-responses of topotecan. Comparable results can be observed from the conventional assays up to T:Q=2:1 (p=0.86). However, further increase in topotecan concentrations beyond the 2:1 ratio did not enhance cytotoxicity. (C) Heat map illustrating cytotoxic effects of the various concentrations of drugs when they were used alone or in combination. Note the dosing ratio 1:1 indicates that cells were exposed to individual drugs for 30 sec each post-electroporation, which is equivalent to treating cells with 330 nM and 500 nM of topotecan and quercetin, respectively, well plates. Error bar represents the standard error of measurements from three independent experiments.

FIG. 4. (A) High performance liquid chromatography (HPLC) profile showing the separation of the unbound Alexa Fluor 488 dye from the conjugated bleomycin. The bleomycin conjugate leaves the column before the unreacted dye. Shaded area indicates the purified conjugate for electroporation experiments. (B) Purified bleomycin-conjugated molecules are introduced into MDA-MB-231 cells using the parallelized micro-vortex assisted electroporation platform. Note that 10 fluorescent images were taken individually from a single experiment and stitched together. Scale bar is 100 μm.

FIG. 5. The number of viable MDA-MD-231 cells linearly correlates with luminescent intensity. Error bars represent the standard deviations of measurements from three independent experiments. Relatively larger standard deviation displayed at the last data point could be attributed by difficulties associated with manually counting a large population of cells.

FIG. 6. A perspective view of an electrode used to create a substantially uniform electric field across traps according to an example embodiment.

FIG. 7A and 7B. Exemplary electrode configurations.

### DETAILED DESCRIPTION

Traditional electroporation drug screening platforms have shown great promise in drug discovery, yet they have limited ability to perform cytotoxicity assays for combination therapies because its stochastic molecular delivery process precludes the determination of synergic drug dosage combinations.

To overcome shortcomings of bulk electroporation, many electroporation systems using microfluidic or nanomaterial platforms have demonstrated reduction in operational costs and reagent consumptions with enhancement in molecular delivery efficiency and viability (Lee et al., 2013; Adamo et al., 2013; Xie et al., 2010; Hur et al., 2010; Kim et al., 2007; Kim et al., 2009; Kotnik et al., 1997; Lee et al., 1997; Lee et al., 2009; Lee et al., 2008; Lin et al., 2009). However, very few systems allow for multi-molecule delivery with controlled dosages besides non-trivial off-chip collection of processed biological samples for downstream analyses. (Xie et al., 2013; Boukany et al., 2011).

Combining the advantages that microfluidics and electroporation offer, direct drug cocktail analysis using the vortex-assisted electroporation platform was demonstrated. The system enables sequential deliveries of precisely controlled amount of multiple molecules into preselected cells, which can be subsequently released for downstream analysis. As a proof of concept, dosage-dependent cytotoxicity of chemotherapeutic drugs and an anticancerous flavonoid was examined when those compounds were used as a single agent or in combination. The platform has a great potential for facilitating comprehensive assessments of drug combinations directly on patients' samples.

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims. In various embodiments, a method provides vortex-assisted microfluidic electroporation utilizing sequential multi-molecule delivery to pre-selected target cells with precise and independent molecular amount and parameter controllability. In one embodiment, at least one of the molecules provides a prophylactic or therapeutic effect when delivered to a cell or mammal, e.g., human, bovine, equine, swine, ovine, caprine, feline, canine or non-human primate. In one embodiment, at least one of the molecules is a chemotherapeutic drug. In one embodiment, at least one of the molecules is a protein, e.g., an antigen. In one embodiment, at least one of the molecules is an antibody or a modified antibody, e.g., a Fv, ScFv or other intrabodies, a monoclonal antibody (mAb) or a humanized antibody, a nucleic acid encoding such an antibody, or a protein conjugate that does not include an antibody. For example, the antibody may be a monoclonal antibody (mAb) conjugated to a chemotherapeutic drug, radiolabeled antibody, including but not limited to antibodies and conjugates such as trastuzumab (Herceptin®), an antibody against the HER2 protein, ibritumomab tiuxetan (Zevalin®), brentuximab vedotin, an antibody that targets the CD30 antigen attached to MMAE, ado-trastuzumab emtansine, an antibody that targets the HER2 protein, attached to cDM1, denileukin diftitox , which is as interleukin-2 (IL-2) attached to a toxin from the germ that causes diphtheria, bevacizumab, a mAb that targets VEGF, cetuximab, an antibody that targets EGFR, OKT3, Muronomab-CD3, ReoPro (Abciximab), Rituxan (Rituximab), Zenapax (daclizumab), Simulect (basiliximab), Synagis (palivizumab), Remicade (infliximab), Mylotarg (gemtuzumab ozogamicin), Campath (alemtuzumab), Humira (adalimumab), Xolair (omalizumab), Bexxar (tositumomab-I-131), Raptiva (efalizumab), Erbitux (cetuximab), Avastin (bevacizumab); Tysabri (natalizumab), Actemra (tocilizumab),Vectibix: (panitumumab), Lucentis (ranibizumab), Soiris (eculizumab), Cimzia (certolizumab pegol), Simponi (golimumab), Itlaris (canakinumab), Stelara (ustekinumab), Arzerra (ofatumumab), Prolia (denosumab), Numax (motavizumab), ABThrax (rasibacumab), Benylsta (belimumab), Yervoy (ipilimumab), Adcetris (brentuximab vedotin), Perjeta (pertuzumab), Kadcyla (adotrastuzumab emtansine), and Gazyva (obinutuzumab). In one embodiment, if one of the molecules to be tested is an antibody, the antibody may be added to the cells of interest without providing an electric field or in the absence of a vortex.

In one embodiment, the cells in the pre-selected population are from a patient, e.g., blood cells. In one embodiment, the cells in the pre-selected population are cancer cells. In one embodiment, the cells in the pre-selected population are stem cells. In one embodiment, different cell types are tested in parallel. In one embodiment, cells in different traps are subjected to an electric field for different lengths of time. In one embodiment, the cells are obtained from a physiological fluid, e.g., blood, urine, peritoneal fluid or pleural fluid.

For example, the cells may be pluripotent cells or are embryonic stem cells other than human embryonic cells or a subset thereof, umbilical cord cells or a subset thereof, bone marrow cells or a subset thereof, peripheral blood cells or a subset thereof, adult-derived stem or progenitor cells or a subset thereof, tissue-derived stem or progenitor cells or a subset thereof, mesenchymal stem cells (MSC) or a subset thereof, skeletal muscle-derived stem or progenitor cells or a subset thereof, multipotent adult progentitor cells (MAPC) or a subset thereof, cardiac stem cells (CSC) or a subset thereof, multipotent adult cardiac-derived stem cells or a subset thereof, cardiac fibroblasts, cardiac microvasculature endothelial cells, or aortic endothelial cells.

In one embodiment, the cells are cancer cells or noncancerous cells from a patient with adrenal cancer, anal cancer, bile duct cancer, bladder cancer, bone cancer, brain/CNS cancer, breast cancer, Castleman Disease, cervical cancer, colon/rectum cancer, endometrial cancer, esophagus cancer, Ewing Family of tumors, eye cancer, gallbladder cancer, gastrointestinal carcinoid tumors, gastrointestinal stromal tumor (GIST), Hodgkin Disease, Kaposi Sarcoma, kidney cancer, laryngeal and hypopharyngeal cancer, leukemia, e.g., ALL AML, CLL, CML, CMML, liver cancer, lung cancer, e.g., Non-Small Cell, Small Cell, Lung Carcinoid Tumor, lymphoma, malignant mesothelioma, multiple myeloma, myelodysplastic syndrome, nasal cavity and paranasal sinus cancer, nasopharyngeal cancer, neuroblastoma, Non-Hodgkin Lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma, skin cancer, e.g., basal or squamous cell, melanoma, or Merkel cell, small intestine cancer, stomach cancer, testicular cancer, thymus cancer, thyroid cancer, uterine Sarcoma, vaginal cancer, vulvar cancer, Waldenstrom Macroglobulinemia, or Wilms Tumor.

Exemplary chemotherapeutics drugs for combination therapy that may be tested in the methods of the invention include but are not limited to alkylating agents including nitrogen mustards such as mechlorethamine (nitrogen mustard), chlorambucil, cyclophosphamide (Cytoxan®), ifosfamide, and melphalan, nitrosoureas which include streptozocin, carmustine (BCNU), and lomustine, alkyl sulfonates, e.g., busulfan, triazines, e.g., dacarbazine (DTIC) and temozolomide (Temodar®) and ethylenimines, e.g., thiotepa and altretamine (hexamethylmelamine), antimetabolites, e.g., 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, Thioguanine; anthracyclines, e.g., Daunorubicin, Doxorubicin (Adriamycin®), Epirubicin, Idarubicin; Anti-tumor antibiotics that are not anthracyclines such as Actinomycin-D, Bleomycin, Mitomycin-C; topoisomerase I inhibitors including topotecan and irinotecan (CPT-11).; topoisomerase II inhibitors including etoposide (VP-16) , teniposide and mitoxantrone; mitotic inhibitors including Taxanes such as paclitaxel (Taxol®) and docetaxel (Taxotere®), Epothilones, e.g., ixabepilone (Ixempra), Vinca alkaloids, e.g., vinblastine (Velban®), vincristine (Oncovin®), and vinorelbine (Navelbine®), and Estramustine (Emcyt®), L-asparaginase, and bortezomib (Velcade®). Examples of targeted therapies include imatinib (Gleevec®), gefitinib (Iressa®), sunitinib (Sutent®) and bortezomib (Velcade®). Other examples include the retinoids, tretinoin (ATRA or Atralin) and bexarotene (Targretin), as well as arsenic trioxide (Arsenox); the anti-estrogens: fulvestrant (Faslodex®), tamoxifen, and toremifene (Fareston®); aromatase inhibitors, e.g., anastrozole (Arimidex®), exemestane (Aromasin®), and letrozole (Femara®), progestins such as megestrol acetate (Megace®); estrogens, anti-androgens: bicalutamide (Casodex®), flutamide (Eulexin®), and nilutamide (Nilandron®); gOtonadotropin-releasing hormone (GnRH), also known as luteinizing hormone-releasing hormone (LHRH) agonists or analogs: leuprolide (Lupron®) and goserelin (Zoladex®). Other agents that may be employed as chemotherapeutics to treat cancer include non-specific immunotherapies and adjuvants (other substances or cells that boost the immune response), such as BCG, interleukin-2 (IL-2), and interferon-alfa. Immunomodulating drugs, for instance, thalidomide and lenalidomide (Revlimid®) and cancer vaccines (e.g., the Provenge® vaccine for advanced prostate cancer).

The methods of the invention may also be employed to determine the effect of combination therapies on other diseases, e.g., diabetes, tuberculosis, leprosy, malaria, and HIV/AIDS, and determine efficacious and synergistic combinations of agents and amounts. Thus, a sample from a patient with diabetes may be employed to determine synergism or antagonism of a combination of anti-hyperglycemic drugs, e.g., including but not limited to metformin, sulfonylureas, meglitinide, PPAR-gamma agents, alpha-glucosidase inhibitors, pramlintide, colesevalem, bromocriptin, thiazolidinediones, or dipeptidylpetidase (DDP)-4 inhibitors, and optionally synergistic amounts of a combination thereof.

A sample from a patient with malaria may be employed to determine synergism or antagonism of a combination of one or more of aminoquinolines, arylaminoalcohols, 8-aminoquinolines, artemisinines, antifolates, antibiotics, inhibitors of the respiratory chain, e.g., atovaquone, proguanil, doxycycline, clindamycin, diaminopyrimidine, sulfonamide, tetracycline, napthoquinine or sesquiterpene lactone, or at least two within each of those classes of agents, and optionally synergistic amounts of a combination thereof. A sample from a patient with leprosy may be employed to determine synergism or antagonism of a combination of clofazimine, dapsone, minocycline, ofloxacin, prednisolone or rifampicin, and optionally synergistic amounts of a combination thereof. A sample from a patient with HIV/AIDS may be employed to determine synergism or antagonism of a combination of one or more of entry inhibitors, fusion inhibitors, reverse transcriptase inhibitors, integrase inhibitors, and protease inhibitors, or at least two within each of those classes of agents, and optionally synergistic amounts of a combination thereof.

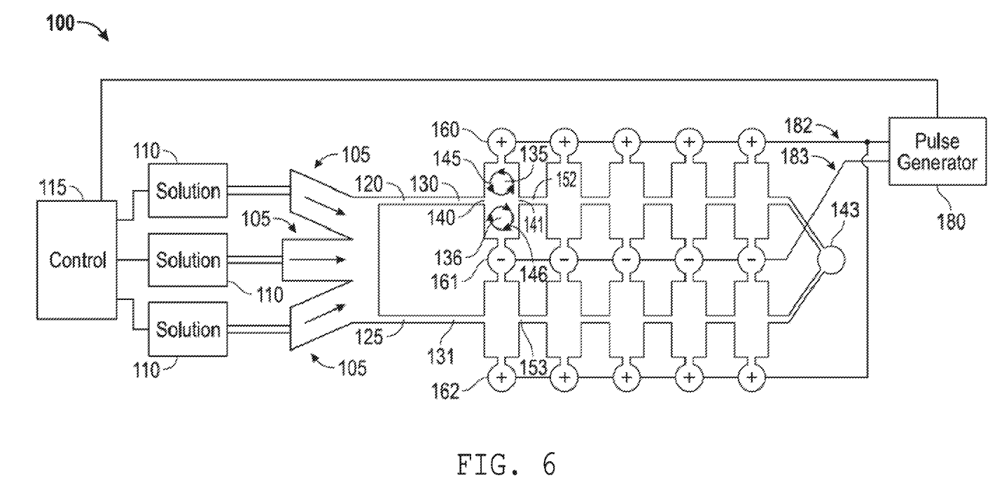

FIG. 6 illustrates an electroporation system generally at 100 in one embodiment useful in carrying out the methods of the invention. System 100 in one embodiment includes multiple inlets 105 coupled to receive solution from multiple sources of solution 110. In various embodiments, the sources 110 are suitable for holding a solution that includes cells of interest, and various solutions containing different molecules of interest. In some embodiments, the solutions may also include solutions for incubation and washing or flushing (such as DPBS) between or after flows of the various solutions. A controller 115 controls the sources 110 to sequentially supply the solutions in a desired manner to the input ports. In one embodiment, the controller comprises a pneumatic flow control system that timely and independently pressurizes individual solution chambers 110 to drive flow through the microfluidic electroporator 100. The controller also controls valves, such as a valve manifold to provide the various solutions in a controlled sequential manner.

The inlets 105 may include coarse filters and are each coupled to at least a first channel 120, and optionally a second channel 125 to provide the solutions to the channels. The first and second channels run substantially parallel to each other in one embodiment and receive the same solutions. Further channels may be included in further embodiments. Each channel may also include an inertial focusing region illustrated at 130, 131 respectively. The inertial focusing regions serve to allow migration responsive to fluid dynamic forces of cells in solution toward walls of the channels.

Multiple chambers, referred to as traps 135, 136 and 138, 138 for example are coupled to the channels 120 and 125 respectively, downstream from the inertial focusing regions 130, 131. In one embodiment, each channel as several pairs of opposing traps that are disposed about a trap entrance 140 and trap exit 141 in each pair of traps. The entrance 140 and exit 141 effectively bisect the pair of traps 135, 136, which form a rectangular structure. The solutions continue to run through the pair of traps via respective inlets and outlets of each pair, and finally exits one or more channel outlets represented at outlet 143 where waste and cells may be collected.

In one embodiment, there are ten traps per channel corresponding to five pairs of opposing traps. Given two channels, that makes for 20 total traps. In further embodiments, up to 80 or more pairs of traps may be formed, equating to 160 or more vortices for trapping cells. The traps and channels may be formed of a polydimethly siloxane (PDMS) sheet in one embodiment from a silicon casting mold formed using common photolithographic processes. The PDMS sheet may be activated (or functionalized), such as by an oxygen cleaner and topped with a glass sheet to enclose the traps and channels. In further embodiments, different materials may be used, such as rigid plastic, allowing higher pressures and hence additional pairs of traps.

The sizes of the traps in one embodiment are selected such that the solution flowing through the channel and traps forms a vortex in each trap as illustrated for example at 145 in trap 135 and at 146 in trap 136. The cells are trapped in the vortices 145, 146 while the solution is flowing when the Reynolds number is greater than approximately 100. The solution keeps flowing through each of the remaining opposed trap pairs separated by sections 152, 153 of the channels. Each trap pair has vortices created, along with trapped cells as the solution flows toward the outlet 143. In one embodiment, each of the traps may contain almost identical populations of cells.

In some embodiments, the forces acting on the cells may cause the cells to be inertially focused to distinct lateral equilibrium positions near the channel walls, such as in inertial focusing regions 130, 131 depending on their biophysical properties (e.g., size and deformability). This particle/cell ordering phenomenon could be resulted from a balance between two counteracting inertial lift forces, namely a wall effect lift and a shear-gradient lift, acting on flowing cells. As inertially focused flowing cells enter the suddenly-expanding traps, also referred to as a electroporation chamber regions, the shear-gradient lift alone induces lateral migration of flowing cells towards the core of microscale vortex since the magnitude of the wall effect lift that had entrained cells at the distinct lateral positions in the straight focusing channel diminishes due to disappearance of channel walls from the vicinity of flowing cells. Since the shear-gradient lift force scales with the third-power of cell's diameter, larger cells are prone to migrate much more rapidly toward the vortex core than smaller cells. Once larger cells migrate close enough to vortices, they remain trapped in the vortices while smaller cells were being flushed out of the device. This gentle and stable cell trapping mechanism allows cascading additional biological assays in situ. The sample injection time may be varied from 20 to 30 seconds at a fixed flow rate of 400 μL/min in order to investigate time-dependent variations in size and number of trapped cells in the electroporation chambers. Further variation of the parameters may occur in further embodiments.

Once a desired number of cells are trapped in the traps by the vortices, the cell solution may be flushed. An optional flushing solution may be used to flush the cell containing solution, followed by a new solution containing molecules of interest. The flow of solutions may be maintained to maintain the vortices and keep the cells trapped in the traps. The cell containing solution is effectively removed from the traps and channels by the new solution. Once a desired concentration of molecules of interest is obtained about the cells in the traps, electrodes 160, 161, 162 are used to create an electric field across the traps, and cause the molecules of interest to transfer the molecules into the cells. Inherently membrane-impermeant molecules can be transferred uniformly across an entire cytosol with a precisely controlled amount.

In one embodiment, electrodes may be coupled to electrical equipment 180 for generating high-voltage short-pulses in the electroporation chambers.

The equipment 180 in one embodiment includes a pulse generator and two electrodes 182, 183 which may be made of platinum or other conductive material. The electrodes 182, 183 may be coupled to electrodes 160, 162, and 161 respectively, which may be directly in contact with a solution in the electroporation traps via ports in the traps. In one embodiment, square wave pulses with magnitude, V, may be varied from 10 V to 200 V for DC and 10 to 100 Vrms for AC field to provide an electric field strength, E =V/Le, applied across the electroporation traps or chambers ranging from 0.1 to 2 kV/cm and from 0.1 to 2 kVrms/cm. The magnitude and duration of applied pulses may be varied simultaneously in order to determine the optimum electrical condition for sequential multi-molecule delivery.

In further embodiments, further solutions with different molecules may be sequentially delivered and optionally transferred into the cells via electrode initiated electroporation. In one embodiment, the electrodes are disposed about ends of the traps in an efficient manner, with traps from different channels that are adjacent to each other sharing a common electrode of a selected polarity, such as negative polarity as shown at 161. Electrodes 160 and 162 are shown as opposite polarity, in this case positive.

The channels and traps in the figures are not necessarily shown to scale. Some example dimensions include the channel having a width such that cells traveling through the channel are approximately 30 percent or greater than the width of the channel. For example, breast cancer cells may be 16 to 20 μm in diameter, resulting in a 40 to 50 μm size channel. The cancer cells in one embodiment may be buffered in a phosphate buffered saline solution such as DBPS, 1×, without Ca²⁺ and Mg²⁺, Cellgro®, Mediatech, USA with a concentration ranging from 1×10⁵ to 1×10⁶ cells/mL. Cell concentration may be varied significantly in further embodiments. The inertial focusing regions may be approximately 0.7 cm or longer in some embodiments to ensure migration of the cells toward walls that facilitate entry of the cells into the vortices. In one embodiment, the traps may have sides (lengths) of between approximately 2 mm to 300 μm, e.g., 1 mm to 500 μm or 1 mm to 400 μm, and are approximately square in cross section. In further embodiments, the side of the trap running with the channel may be longer than the distance or depth that the trap extends away from the channel.

In one example embodiment, the inertial focusing channel may have dimensions of (L=4.5 cm, W=40 μm, and H=60 μm). The electroporation chambers or traps have an approximately square shape with each edge, W_{c}=400 μm. The dimensions may be varied in further embodiments, with a length of the traps along the length of the channel being longer than the width while still maintaining the ability to maintain a vortex flow in the traps via the solutions. The height of the traps may be substantially the same as the height of the channel to maintain ease of manufacturing. The height may vary in further embodiments.

In one embodiment, the structure described is able to trap vertebrate, e.g., mammalian, cells in the traps, and may deliver molecules having a wide range of molecular weights (e.g., 3,000 to 70,000 Da, neutral or anionic molecules) to the trapped cells. The cells and different molecules may be delivered sequentially, such that for each molecule, the electric field may be tailored for electroporation of each cell and molecule combination. A broad range of molecules commonly used prophylactically or therapeutically may be employed. In some embodiments, a broad range of molecules, regardless of their electrical charges (e.g., anionic or neutral), can be introduced into the cells using identical electrical parameters.

In one embodiment, system 100 may be an on-chip microscale electroporation system that enables sequential delivery of multiple molecules with precise and independent dosage controllability into pre-selected nearly identical populations of target cells. The system 100 provides the ability to trap cells with uniform size distribution contributing to enhanced molecular delivery efficiency and cell viability. Additionally, the system 100 may provide real-time monitoring ability of the entire delivery process, allowing timely and independent modification of cell- and molecule-specific electroporation parameters. A precisely controlled amount of inherently membrane-impermeant molecules may be transferred into cells, such as human cancer cells, by varying electric field strengths and molecule injection durations.

In one embodiment, fluidic forces tend to push larger particles, such as cells and larger molecules outward from the direction of flow and cause them to be more likely to be trapped in the vortices than smaller particles. Smaller particles are also less likely to be trapped even if they enter the vortices, making it effective to flush out solutions used to carry the larger particles once trapping and electroporation have been performed.

In one embodiment, the system has a single channel having a plurality of serially opposed traps. Cells (a first set) are introduced and distributed to the traps via the single (first) channel and cells are detained in a vortex in the traps. In one embodiment, delivering cells of interest in solution to multiple traps via the channel connecting the traps includes providing a first solution to the channel via an inertial focusing region to cause the cells of interest to move close to the sides of the channel via fluidic forces. In yet a further embodiment, the channel breaks into multiple channels, each having opposing pairs of traps disposed along a length of the channels. A first solution having a first molecule of interest at a selected concentration is introduced to the channel and an electric field is applied for a defined (first) period of time. The first solution is removed and optionally replaced with a wash solution, while the cells remain detained in the traps. A second solution having a second molecule of interest at a selected concentration is introduced to the channel and an electric field is applied for a defined (second) period of time. The first and second periods of time may be the same or different. The cells are collected and the effect of the two molecules on the cells is determined. Optionally, after the first set of cells is collected, a second set of cells is introduced to the single channel and the same two molecules are introduced to the cells but at least one is at a different concentration, or the electric field is applied for a different length of time, and the effect of the two molecules on the cells is determined. In one embodiment, cytotoxicity or viability of the collected cells is determined. In one embodiment, the cells are infected with a virus and the anti-viral activity of the two molecules is determined. In one embodiment, the cells are infected with a bacterium or parasite and the anti-bacterial or anti-parasitic activity of the two molecules is determined.

In one embodiment, the system has at least two distinct channels. In one embodiment, cells are introduced to the two channels and one or more inertial focusing regions detain the cells in the traps. In one embodiment, different cells are introduced to each of the two channels. In one embodiment, the two channels are employed to deliver different concentrations or combinations of the molecules. For instance, the cells in traps in a first channel are exposed to drug 1 at concentration 1 for time period 1 and to drug 2 at concentration 2 for time period 2, and the cells in traps in a second channel are exposed to drug 1 at concentration 1 for time period 3 and to drug 2 at concentration 2 for time period 2 or time period 4. In one embodiment, time period 1 and time period 2 are the same. In one embodiment, time period 3 and time period 4 are the same.

In one example method, each solution may be injected into the device sequentially at an operating pressure of, for instance, 40 psi (equivalent to a flow rate of 400 μL/minute) using the flow control 115. Operating parameters are adjusted depending on materials in the system and geometries, e.g., higher pressures may be needed to create and maintain vortices when plastic materials are employed. Prior to the cell solution injection, the washing solution may be injected for >1 minute in order to prime the flow speed required for stable microscale vortex generation. Then, target cells are isolated into the electroporation chambers using the microvortex trapping mechanism simply by switching the active solution port from the washing solution to the cell solution.

Once the desired size and number distribution of trapped cells has been achieved, solution is rapidly exchanged to the washing solution in order to remove non-trapped contaminating cells from the entire device without disturbing orbits that trapped cells created. After the device is flushed for 20 seconds, solutions containing the first and the second molecule to be transferred (e.g., fluorescent nucleic dyes or fluorescent protein plasmids) may be sequentially injected.

Single or multiple short pulses of high electric field may be applied promptly after injection of each molecular solution had been initiated. The magnitude, duration and number of square pulses as well as incubation time could be individually varied depending on cellular and molecular types to have better experimental outcomes. Upon completion of molecular delivery using electroporation, the processed cells may be re-suspended in the washing solution and released from the device for downstream analysis by simply lowering the operating pressure to below 5 psi, followed by final device flushing step at 40 psi for 10 seconds.

The average size and size-uniformity of the captured cells in the electroporation chambers increases with increasing cell solution injection time. While the average diameter of trapped cells, Dave, may be about 20 μm with a coefficient of variation (CV) of 40% shortly after the cell solution injection has been initiated (t=10 s), the Dave may increase to 32 μm with a reduced CV of 25% at t=30 seconds. For a square electroporation chamber geometry (Each side=400 μm), the average number of cells processed in each run may be about 20 when the maximum size and uniformity have been achieved and the number of cells is maintained identical throughout the course of the entire electroporation processes.

The ability to trap cells with a uniform size distribution positively contributes to having better molecular delivery efficiency and cell viability because the cell size distribution has significant implication for membrane permeabilization. When cells are exposed to an extracellular electric field, E, transmembrane potential, ΔVm=f E D cos θ, is induced in a cell. Here, f is the weighting factor, which is the measure of how cells contribute on the applied electric field distribution, and D and θ are the cell diameter and the polar angle measured with respect to the external field, respectively. In order to transiently permeabilize cells without cell lysis, ΔVₘ should exceed the transmembrane potential, ΔVₛ, but remain below the irreversible electroporation threshold, ΔV_{c}. For mammalian cells, ΔVₛ is reported to range between 200 mV and 1 V, depending on pulse duration, while ΔV_{c} is expected to be greater than 1 V.

Previous electroporation tests performed with cell lines that have a large size variation are reported to have low electroporation efficiency and viability since samples with higher heterogeneity in size are expected to have more number of cells having induced ΔVₘ that falls outside of the optimum range for successful transient permeabilization. Therefore, the current system's ability to pre-isolate cells with higher homogeneity in size may minimize undesirable consequences associated with a large cell size variation.

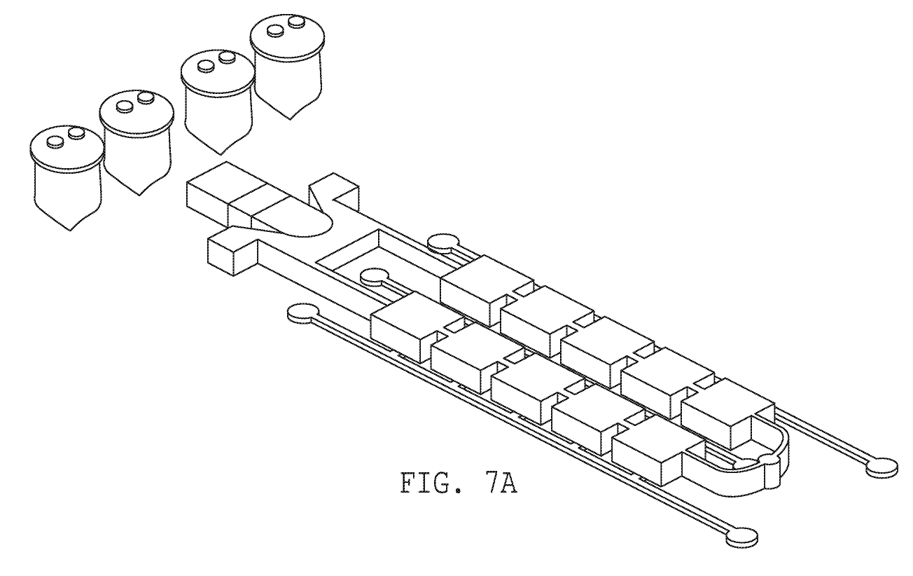

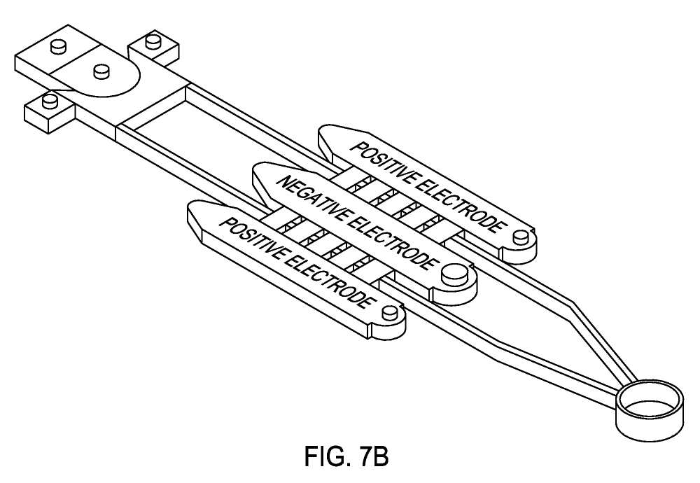

In one embodiment, the amount of transferred molecule proportionally increases with the electric field strength when cells are exposed to the identical molecular amount (for example, solution injected into the device for 40 s and processed cells were washed with molecule-free DPBS for 10 s. An Eᵢ=0.4 kV/cm is sufficient in one embodiment to initiate the delivery of molecules to the cells and E exceeding 2.0 kV/cm may result in cell lysis. Similarly, the molecular transfer dosage can be monotonically increased by increasing solution injection time with the set electric field strength, Eₒ=0.8 kV/cm. Eₒ is one of the optimal conditions, where processed cells exhibited high viability (83%) and electroporation efficiency (70%). Electroporated cells' molecular uptake may be initiated after cells are exposed to approximately 800 ng of molecules (equivalent to the solution injection time, t=5 s). For different cells, similar trends may be observed with slightly higher Eᵢ=0.6 kV/cm and Eₒ=1.0 kV/cm. Different effective electric field values may depend on differences in membrane properties or average cell diameter. The system's rapid solution exchange scheme combined with real-time monitoring ability enabled the precisely and independently control and optimization of each transferred molecular dosage simply by varying incubating-duration and/or electric field strength. The electric field may be orthogonal to flow (see FIG. 7), e.g., the electrodes may be placed along the width, height or length of the traps.

For each solution applied after the cells are introduced to the traps, vortex flow is maintained in the traps, until the cells are collected for analysis. In further embodiments, the electric field may be adjusted or tailored to optimize delivery of each different molecule, especially if the molecules have quite different molecular sizes or electrical properties.

The invention will be further described by the following non-limiting example.

### EXAMPLE

### Materials and Methods

### Cell Preparation

Metastatic breast cancer cells, MDA-MB-231 (HTB-26, ATCC), were plated at a concentration of 1×10⁵ cells/mL in a volume of 10 mL per a T75 tissue culture flask (CELLSTAR®, Greiner Bio-One, USA) in Leibovitz's L-15 Medium (Cellgro®, Mediatech, Inc., USA) supplemented with 10% (v/v) fetal bovine serum (FBS, Gibco®, Life technologies, USA) and 1% penicillin streptomycin (Sigma-Aldrich Co., USA). These cells were incubated in a humidified incubator at 37° C. with 0% CO₂ environment. Cells were harvested for experiments 2 days after the seeding by treating them with 0.25% trypsin-EDTA for 2 minutes. The cells were pelleted by centrifuging for 5 minutes at 200×g and resuspended in the growth media to have a final concentration of 5×10⁵ cells/mL.

### Device Design and Fabrication

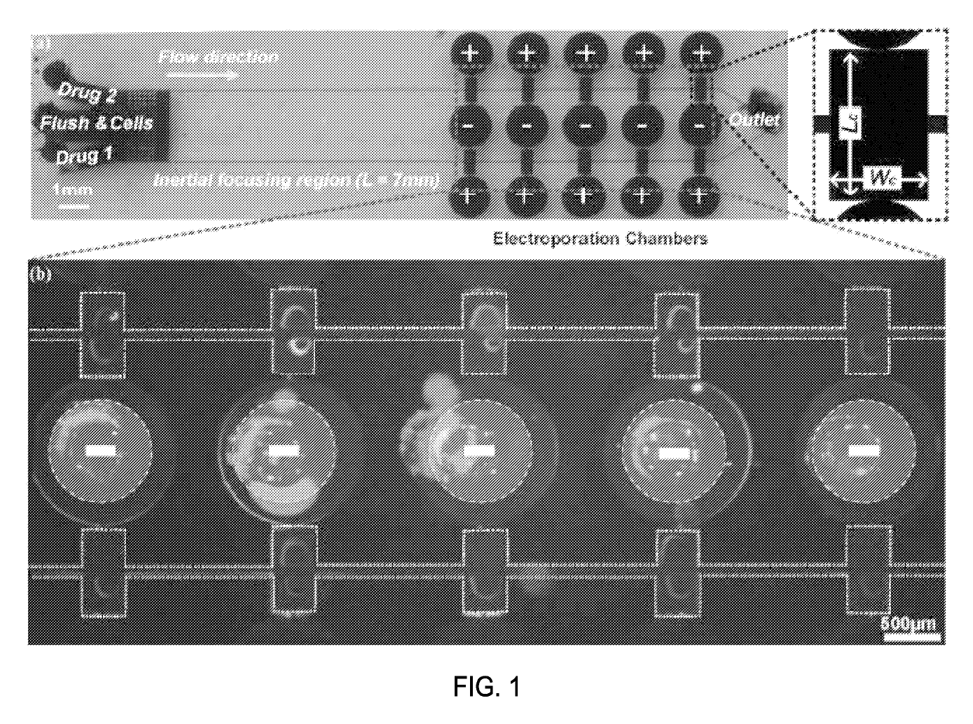

A micro-vortex assisted electroporation platform was parallelized in order to enhance the throughput without sacrificing any of its merits. The parallelized microfluidic electroporator consists of an inlet with multiple solution injection ports and coarse filters, two parallel inertial focusing channels (L=7 mm, W=40 μm, and H=70 μm), and an outlet where two straight inertial focusing channels merge (FIG. 1A). Individual straight channels consist of 5 electroporation chambers in series and the electroporation chambers are placed 800 μm apart (Wc=400 μm and L_{c}=840 μm). Individual electroporation chambers have two via holes for aluminum electrodes and two transversally adjacent chambers share the hole for negative electrodes. 2-dimensional projection of the device was designed using AutoCAD (Autodesk, Inc., USA) and the CAD file with micro-patterns was converted to a GDSII file using LinkCAD. The micro-patterns were written on a 5″×5″ photomask blank using a laser mask writer (Heidelberg Mask Writer, DWL-66). The mask was developed by following the manufacturer's protocol. The casting mold was then fabricated using negative photoresist (KMPR 1050, Microchem, USA) by following conventional photolithography procedures. The heights of fabricated microstructures were measured using a surface profilometer (Dektak 6M, Veeco, USA). Polydimethysiloxane (PDMS, Sylgard® 184 silicone elastomer kit, Dow corning, USA) replicas were generated by following the soft lithography techniques (Na et al., 1995). PDMS was degassed for 30 minutes and cured for 2 hours in an oven maintained at 70° C. Cured PDMS replicas were delaminated from the mold, and solution injection ports, an outlet, and electrode insertion holes were created by using a pin vise (Pin vise set A, Syneo). Microchannels were enclosed by bonding PDMS replicas to glass slides after oxygen treatments (Technics Micro-RIE, USA).

### Electroporation of Target Cells for Drug Delivery

The system utilized the pneumatic flow control unit and the electrical equipment that we previously developed. Their detailed specifications and visualized operational protocols (Yun et al., 2013; Vickers et al., 2014) can be found elsewhere. In brief, the flow control unit independently and promptly pressurizes individual solution vials for rapid solution exchanges through the microfluidic system while the electrical equipment generates high-voltage short pulses across the electroporation chambers on demand. Prior to electroporation experiments, a 15-pin aluminum electrode array (Vickers et al, 2014) and an outlet PEEK tube were inserted into the microfluidic device, and the electrical equipment connected to the electrode array. Solution vials, individually containing cells, drugs and a growth media for flushing, were mounted into the pneumatic flow control unit, and PEEK tubes from each vial inserted into designated inlet ports in the microfluidic electroporator. The system was flushed with the growth media at the operating pressure of 40 psi (equivalent to a flow rate of 400 μL/minute) for 90 seconds prior to the target cell injection step in order to prime the flow speed required for cell trapping (Hur et al., 2011). Once the desired size and number distribution of trapped cells were attained in each cell trapping vortex, not-trapped cells were removed from the device by switching the active solution port from the cell solution to the flush solution. Five short pulses with the magnitude, V=100 V, and the pulse width, Δt=30 ms, were applied promptly after the first drug solution was injected into the device. the resulted electric field strength across the electroporation chamber was E =V/Lₑ=0.7 kV/cm. Here, Lₑ=1.5 mm, is the distance between the positive and negative electrodes that are in contact with the flowing solution. The magnitude, V, the width, Kt, and the frequency of applied electrical pulses were monitored in real-time using an oscilloscope (Agilent, USA). Drug delivery doses were varied by exposing trapped cells to a single drug for incubation durations of 30, 60 and 120 seconds in continuous flow. For dual drug delivery tests, trapped cells were sequentially incubated in individual drugs with various dosage combinations by systematically changing treatment duration ratios of two drugs (e.g., 30:30 seconds, 30:60 seconds or 30:120 seconds). Upon completion of the drug delivery process, treated cells were suspended in the growth media and released from the device for downstream analysis by lowering the operating pressure to 30 psi. Collected cells were seeded in 96 well plates and cultured for 24 to 48 hours prior to the cytotoxicity assays. The durations for which cells were cultured prior to the cytotoxicity assays were determined based on conditions previously reported for tested drugs (bleomycin, Todoronc et al., 2009; gemcitabine, Wang et al., 2012; topotecan and quercetin, Akbas et al., 2005).

### Real-Time Molecular Delivery Visualization

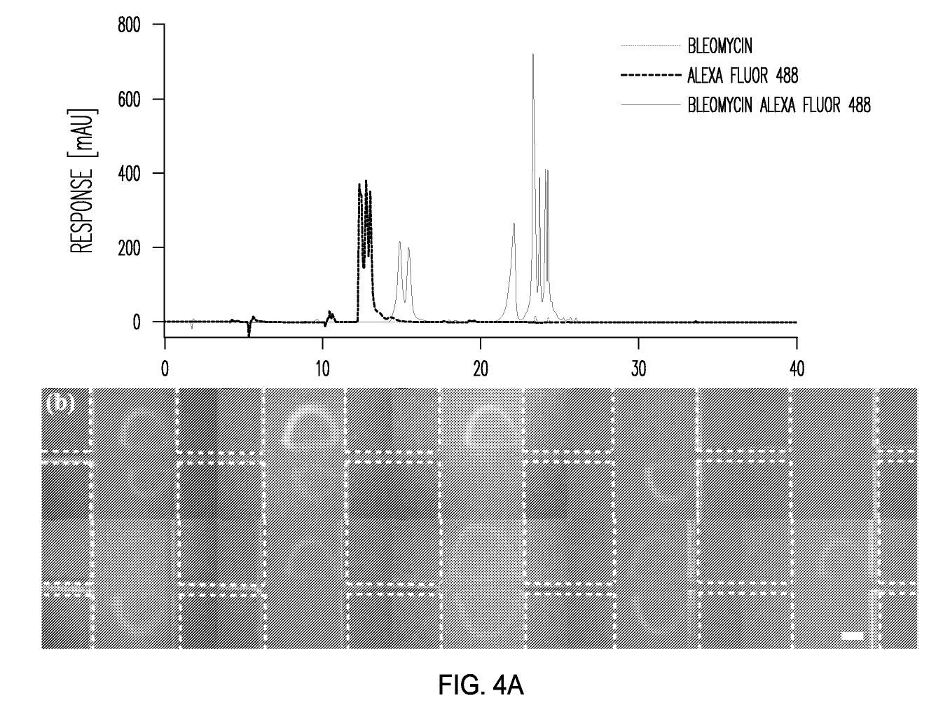

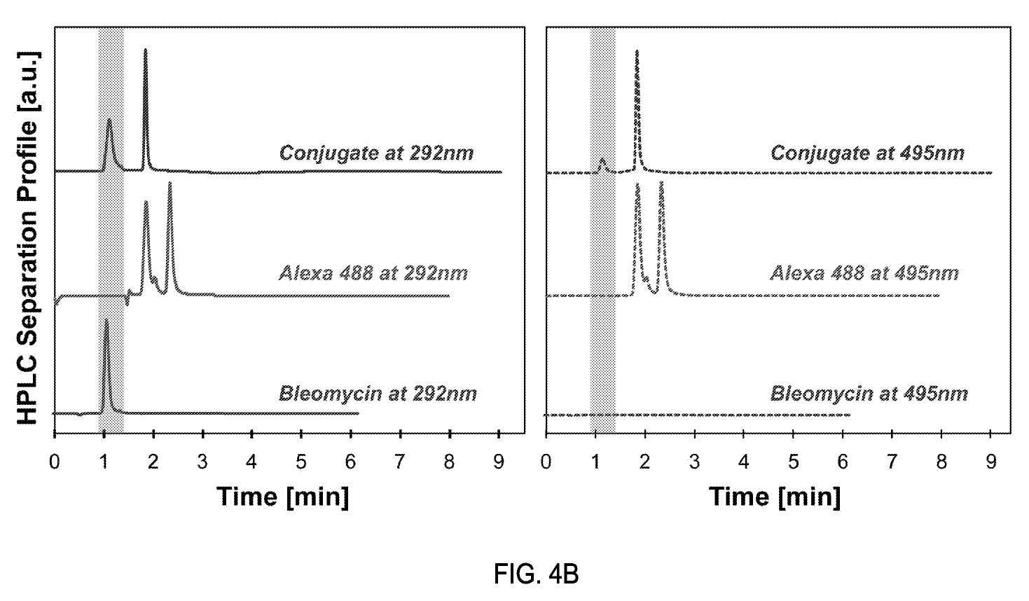

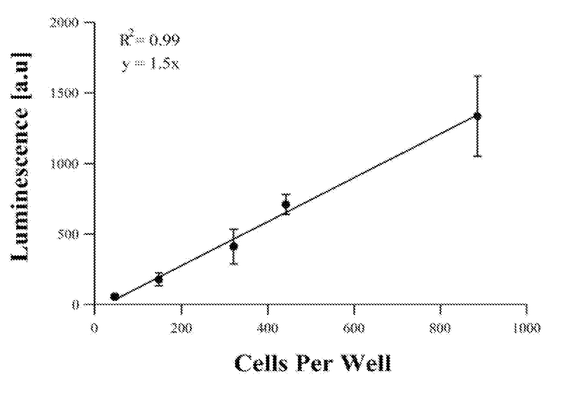

The nucleic acid fluorescent dyes propidium iodide (PI) and YOYO®-1 (Life Technology, USA) were used to visualize intracellular delivery of molecules in real-time, allowing prompt determination of optimum electrical parameters and electroporation efficiencies. In addition, 100 μM of bleomycin fluorescently labeled with Alexa-Fluor® 488 (Life Technology, USA) was used to identify electrical parameters required for successful drug delivery (see, FIG. 4B). Bleomycin was conjugated to fluorophores by following manufacturer's protocol. Unreacted residual dye molecules were removed by performing high performance liquid chromatography (HPLC, Agilent 1100 Series), using methanol and acetonitrile as solvent A and B, respectively (see, FIG. 4A).

### Drug Preparation

Dose responses of metastatic breast cancer cells were tested using chemotherapeutic drugs, bleomycin, gemcitabine and topotecan, and an anticancerous flavonoid, quercetin, in order to illustrate the drug screening capabilities of the proposed platform (Sigma-Aldrich Co., USA). Conventional drug screening assays utilizing well-plates were conducted in parallel to evaluate the performance of the proposed microfluidic system. For the microfluidic electroporation drug screening assays, the delivery dosages were varied by changing incubation durations (e.g., 30, 60 and 120 seconds) at a flow rate of 400 JL/min where the concentrations of gemcitabine, bleomycin, topotecan and quercetin in the injection vials were fixed at 237 μM, 56 nM, 240 nM and 66 nM, respectively. For control experiments using the conventional method, the concentrations of drugs were determined such that cells would be exposed in a well plate for 24 hours to the drug amount that is identical to those used for the microfluidic counterpart. That is, viability of cells incubated in 210, 420 and 840 μM of gemcitabine using the conventional assay were compared to that of cells treated with the drug for 30, 60 and 120 seconds, respectively, in the microfluidic device. Similarly, cells were incubated in bleomycin (60, 110 and 200 nM), topotecan (330, 650 and 1300 nM) and quercetin (0.5, 0.9 and 1.8 μM) for the control experiments.

### Cytotoxicity Determination

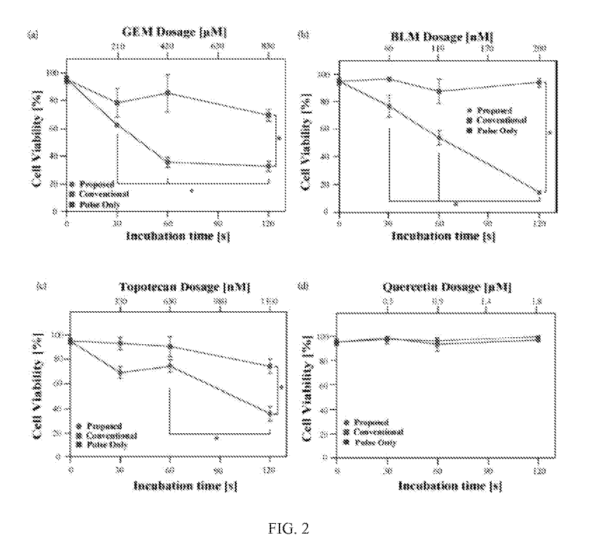

The CellTiter-Glo Luminescent Cell Viability Assay (Promega, USA) was used to determine the cytotoxicity of tested drugs. First, the correlation between the luminescent intensity and the number of viable cells for the tested cell line was established from luminescence measurements from 96 well plates containing known-quantity of viable cells (LuMate, Model 4400) (see ESI FIG. 2 for calibration curve). For cytotoxicity studies on cells treated with the drugs, all cells present in the well, regardless their viability, were manually counted prior to adding the CellTiter-Glo reagent to the well. The number of viable cells was determined by identifying the corresponding number of viable cells represented by the measured luminescent intensity from the calibration curve. Viability was determined by taking the ratio between the number of viable cells and the total number of treated cells presented in the well. The results from the combination treatments were analyzed to determine whether the combination was synergistic, additive, or antagonistic by calculating the combination index (CI) according to the Chou-Talalay method (Chou et al., 1983) using the Compusyn software (Combosyn Inc., USA).

### Results and Discussion

In this work, a parallelized microfluidic electroporation platform was utilized for direct drug cocktail screenings. This platform renders a 10-fold enhancement in throughput in addition to all the merits that the previous single-chamber system provided (Yun et al., 2013). The system's unique merits remaining uncompromised through parallelization include (i) superior viability of processed cells, (ii) on-demand injection of single substances, eliminating unforeseen adverse effects associated with drug-drug interaction prior to delivery and (iii) sequential multi-molecule delivery with precise and independent dosage control and high efficiency. The system takes advantage of the microvortex-assisted cell trapping mechanism (Hur et al., 2011) to pre-select identical population of cells with a uniform size distribution, hereby allowing less variation in electroporation efficiency and enhancement in viability per given electric field strength (Gehl, 2003). Previously, molecules ranging from small nucleic acid dyes to large naked plasmids were shown to be introduced sequentially into pre-selected populations of cells as they orbit in the vortices (Yun et al., 2013). Parallelization of this platform did not interfere with the sequential molecular delivery process as we confirmed through sequential delivery of the nucleic acid dyes, PI and YOYO-1 into cells trapped in the 10-electroporation chambers (FIG. 1B). In a similar manner, multiple drug compounds with varied concentration ratios were delivered into cells orbiting in the electroporation chambers.

To evaluate the system's applicability toward drug screening assays, initially the dose response characteristics of gemcitabine and bleomycin (FIG. 2A and B), which have been studied extensively for electro-chemotherapy for cutaneous metastasis were assessed (Jaroszeski et al., 2000; Wang et al., 2012). Consequently, these drugs are ideal candidates for comparative studies of the microfluidic and the conventional wellplate assay because their cytotoxic effects and optimum dosages have been well-documented. Cells were treated with these drugs as single entities in well plates exhibited less significant dosing effects compared to those treated by the microfluidic device (FIG. 2). This could be attributed by the non-permeant nature of the drugs (Jaroszeski et al., 2000). Correspondingly, higher IC₅₀ values for both drugs were obtained from the conventional assays (Table 1), suggesting that direct injection of drugs into the cytosol induces higher cytotoxicity even though identical drug amounts were accessible in the incubating solution.

TABLE 1
IC₅₀ values evaluated experimentally for both the
microfluidic and conventional assay.

| Chemotherapeutic | | Conventional | Proposed |
|---|---|---|---|
| Drugs | Literature IC₅₀ | IC₅₀ | IC₅₀ |
| Gemcitabine | 340 μM^{†} | 2000 μM | 302 μM |
| Bleomycin | 12 μM^{†} | 20 μM | 0.12 μM |
| Topotecan | 1.1 μM^{†} | 2 μM | 1.2 μM |

^{†}Previously reported IC₅₀ values for tested drugs on various cell types were included for comparison. IC₅₀ values for gemcitabine, bleomycin and topotecan have been reported for Ca-27 squamous carcinoma (Wang et al., 2012), MC38 colon cancer cell line (Kuriyoma et al., 2000), and MDA-MB-231 (Akbos et al., 2005), respectively.

The dose effects of a chemotherapeutic drug, topotecan, and a flavonoid with anticancer properties, quercetin, were investigated as single agents and in combination. Previously, the combination of these two substances as reported to have synergistic effects because quercetin enhances cytotoxicity of chemotherapeutic drugs only in cancerous cells, suppressing side effects of the toxic drug (Akbas et al., 2005; Staedler et al., 2011). As a single agent, topotecan dose-dependently reduced survival of cells processed using either drug-screening platforms (FIG. 2C). The fact that cells treated using the microfluidic platform responded more sensitively to the drug suggests that the proposed microfluidic assay can be used to identify effectiveness of drugs with subtle variation in the concentrations. Quercetin alone, on the other hand, exhibited negligible cytotoxic effect, even at a high concentration, on cells processed with either system (FIG. 2D), in a good agreement with previous studies (Staedler et al., 2011; Chen et al., 2010). No apparent cytotoxic effect on cells treated with atoxic quercetin using the current system further confirms that the dose responses of tested chemotherapeutic drugs were not altered by the electroporation and solely represent actual cytotoxic effects of delivered drugs.

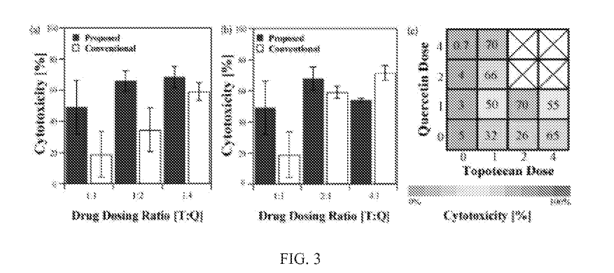

For sequential dual molecular delivery experiments, cells were first exposed to topotecan and quercetin for the identical duration (i.e., 30 seconds incubation in solutions with 240 nM and 66 nM of topotecan and quercetin, respectively, see FIG. 3A). More than 3-fold reductions in IC₅₀ values obtained from both assays suggest that the addition of quercetin augments the cytotoxic effects of topotecan (Table 1). This trends were evident at all combination ratios where the fixed concentration of topotecan was used (T:Q=1:1, 1:2 and 1:4). The combination index (CI) was computed for all tested combinations in order to quantitatively evaluate drug combinatorial effects (Table 2).

TABLE 2
Combination index (CI) value for the combination therapy
of topotecan (T) and quercetin (Q) at various ratios.

| Drug Combination | | |
|---|---|---|
| Ratio [T:Q] | Conventional CI | Proposed CI* |
| 1:1 | 0.32 | 0.34 |
| 2:1 | 0.12 | 0.32 |
| 4:1 | 0.29 | 1.11 |
| 1:2 | 0.15 | 0.17 |
| 1:4 | 0.04 | 0.15 |

*Note that the dose response value of quercetin at 120 second exposure was excluded from CI value determination because dose-independent and negligible cytotoxicity of quercetin resulted in illogical and negative coefficient of determination, R², for the dose response curve.

In such analysis, CI<1 implies synergy, CI=1 represents additive effect, and CI>1 corresponds to antagonism (Chou et al., 1983). Cytotoxicity assays assessed using both platforms illustrated that the drug combinations work in synergy (CI<1) at all tested ratios (FIG. 3A). This trend of increased drug cytotoxicity continues as the dosage of topotecan increases while that of quercetin remains unchanged (T:Q=2:1). The drug ratios, T:Q, being 1:1 and 2:1 were found to induce synergistic effects for both assays. Interestingly, the antagonistic effect (CI=1.11) was detected only with the microfluidic assay at T:Q=4:1 whereas the results from the conventional assay implied the synergic effect of the combination (CI=0.29). Such disparity could have originated from the fact that apparent drug amounts delivered into cytosols by the microfluidic system were much greater than those by the well plate assay. Presumably, a threshold beyond which cytotoxicity is independent of drug concentrations has been attained for the microfluidic assay. It is also possible that the proposed system can identify elusive antagonistic drug combinations, which are not detectable otherwise. Conclusion

A microscale vortex-assisted electroporation platform was implemented for direct analysis of drug cocktails. Contrary to bulk electroporation systems, this platform did not adversely affect cell viability, suggesting that cellular responses observed from cytotoxicity assays solely represent actual effects of the drug cocktail. Through quantitative single and drug combination analyses, the combinational dosages of the chemotherapeutic drug and the flavonoid inducing synergetic and antagonistic effects were identified. The system enables drug screenings for development of personalized medicines when it is integrated with an on-chip cell purification system (Sollier et al., 2014).

### REFERENCES

Adamo et al., Anal. Chem., 85:1637 (2013).

Akbas et al., J. Surg. Res., 125:49 (2005).

Al-Lazikani et al., Nat. Biotechnol., 30:679 (2012).

Booth et al., J. cancer Immunol. Immunother., 58:1229 (2009).

Boukany et al., Nat. Nanotechnol., 6:747 (2011).

Cemazar et al., Br. J. cancer, 84:565 (2001).

Chen et al., Life Sci., 87:333 (2010).

Chou et al., Trends Pharmac. Sci., 4:450 (1983).

Chou, Pharmacol. Rev., 58:621 (2006).

Du et al., Anal. Chem., 85:6740 (2013).

Gehl, Acta Physiol. Scand., 177:437 (2003).

Gothelf et al., cancer Treat. Rev., 29:371 (2003).

Hur et al., Biomicrofluidics, 5:022206 (2011).

Hur et al., Lab on a Chip, 10:274 (2010).

Jaroszeski et al., Anti-cancer Drugs, 11:201 (2000).

Kim et al., Biosens. Bioelectron., 22:3273 (2007).

Kim et al., Nature, 461:649 (2009).

Kolishetti et al., Proc. Natl. Acad. Sci. USA, 107:17939 (2010).

Kotnik et al., Bioelectrochem. Bioenerg., 43:285 (1997).

Lee et al., Anal. Chem., 86:535 (2013).

Lee et al., Crit. Rev. Ther. Drug Carr. Syst., 14:173 (1997).

Lee et al., Integr. Biol., 1:242 (2009).

Lee et al., Lab on a Chip, 8:224 (2008).

Lin et al., Biosens. Bioelectron., 25:388 (2009)

Lundholt et al., J. Biomol. Screen, 8:566, (2003).

Mendez-Ortega et al., Br. Med. J., 326:61 (2003).

Meng et al., PLoS One, 5:e14124 (2010).

Nakamura et al., J. Dev. Growth Diff., 55:15 (2013).

Orlowski et al., Biochem. Pharmacol., 37:4727 (1988).

Sollier et al., Lab on a Chip, 14:63 (2014).

Staedler et al., cancer Chemother. Pharmacol., 68:1161 (2011).

Todorovic et al., Radiol. Oncol., 43:264 (2009).

Vickers et al., JoVE _____:_____ (2014).

Wang et al., PLoS One, 7:e43940 (2012).

Wu et al., BMC Syst. Biol., 4:S7 (2010).

Xia et al., Angew. Chem.-Int. Edit., 37:551 (1998).

Xie et al., ACS Nano, 7:4351 (2013).

Xu et al., BMC Syst. Biol., 6:1 (2012).

Yun et al., Lab on a Chip, 13:2764 (2013).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method comprising:
maintaining a vortex flow in multiple traps having cells of interest;
providing a first molecule of interest to the traps and providing an electric field across the traps for a first period of time allowing for electroporation of the first molecule of interest into the cells of interest in the traps;
providing a second molecule of interest to the traps and providing an electric field across the traps for a second period of time allowing for electroporation of the second molecule of interest into the cells of interest in the traps; and
determining whether the combined effect of the first and second molecules on the cells is synergistic, neutral or antagonistic.

2. The method of claim 1 wherein cytotoxicity, anti-viral activity, anti-parasitic activity or anti-bacterial activity is determined.

3. The method of claim 1 wherein the first molecule of interest, the second molecule of interest, or both, inhibit or treat cardiovascular disease or diabetes.

4. The method of claim 1 wherein the first molecule of interest, the second molecule of interest, or both, inhibit or treat cancer.

5. A method comprising:
maintaining a vortex flow in multiple traps having cells of interest;
providing a first molecule of interest to the traps and providing an electric field across the traps for a first period of time allowing for electroporation of the first molecule of interest into the cells of interest in the traps;
providing a second molecule of interest to the traps with the cells electroporated with the first molecule and providing an electric field across the traps for a second period of time allowing for electroporation of the second molecule of interest into the cells of interest in the traps;
providing a third molecule of interest to cells of interest in the traps and providing an electric field across the traps for a third period of time allowing for electroporation of the third molecule of interest into the cells of interest in the traps;
providing a fourth molecule of interest to the traps with the cells electroporated with the third molecule of interest and providing an electric field across the traps for a fourth period of time allowing for electroporation of the fourth molecule of interest into the cells of interest in the traps; and
determining the combined effect of the first and second molecules on the cells electroporated for the first and second periods of time and the combined effect of the third and fourth molecules on the cells electroporated for the third and fourth periods.

6. The method of claim 5 wherein the first and third molecules are the same but the first and third periods of time are different.

7. The method of claim 5 wherein the second and fourth molecules are the same but the second and fourth periods of time are different.

8. The method of claim 1 wherein a first fluid solution containing the cells of interest has a Reynolds number of greater than 100 to create the vortex flow in the traps.

9. The method of claim 8 wherein a second fluid solution containing the first molecules of interest is introduced to the traps while maintaining the vortex flow and removing the first fluid solution.

10. The method of claim 1 wherein the electric field across the traps is substantially uniform.

11. The method of claim 9 wherein a third fluid solution containing the second molecule of interest is introduced to the traps while maintaining the vortex flow in the traps following electroporation of the first molecule of interest.

12. The method of claim 1 wherein the cells of interest are collected prior to determining the effect.

13. The method of claim 1 wherein the cells of interest are cancer cells, primary cells or cells from a human patient.

14-15. (canceled)

16. The method of claim 1 wherein the first molecule of interest or the second molecule of interest is a chemotherapeutic drug.

17. The method of claim 16 wherein the first molecule of interest or the second molecule of interest is a MEK inhibitor, a BRAF inhibitor, an ERK inhibitor, or an EGFP inhibitor.

18. The method of claim 1 wherein the cells of interest are stem cells.

19. The method of claim 18 wherein the cells of interest are induced pluripotent stem cells.

20. The method of claim 1 wherein the cells of interest are from a physiological fluid sample of a patient.

21. The method of claim 20 wherein the fluid sample is a blood, urine, pleural fluid or peritoneal fluid sample.

22. The method of claim 5 wherein the cells electroporated for the first and second periods of time are different than the cells electroporated for the third and fourth periods of time.
